(19) United States
(12) Patent Application Publication
(10) Pub. No.: US 2002/0058961 A1
(43) Pub. Date: May 16, 2002

(54) Catheter

(76) Inventors: Amiel R. Aguilar, San Carlos, CA (US); Robert K. Deckman, San Bruno, CA (US); Jeffery L. Emery, San Mateo, CA (US); Daniel E. Francis, Stanford, CA (US); Kurt D. Sparks, Palo Alto, CA (US)

Correspondence Address: PERKINS COIE LLP, P.O. BOX 2168, MENLO PARK, CA, 94026, US

(21) Appl. No.: 09/981,526

(22) Filed: Oct. 16, 2001

Related U.S. Application Data
(60) Provisional application No. 60/241,134, filed on Oct. 16, 2000.
(60) Provisional application No. 60/245,343, filed on Nov. 1, 2000.
(60) Provisional application No. 60/268,264, filed on Feb. 12, 2001.
(60) Provisional application No. 60/268,647, filed on Feb. 13, 2001.

(51) Int. Cl. A61M 29/00

(52) U.S. Cl. 606/198

(57) ABSTRACT

Embodiments of a catheter for intravascular procedures are described. The embodiments described include a catheter with various elements arranged about a central axis. The elements include an inner shaft, an outer shaft, and an actuation mechanism. In one embodiment, the inner shaft forms the actuation mechanism, and includes a flexible hypotube. The flexible hypotube further forms a lumen that can accommodate, for example, a guidewire. The actuation mechanism actuates, or deploys, a working element at the distal end of the catheter. In one embodiment, the working element includes a tissue spreading member for disrupting an occlusion. In one embodiment, the catheter includes an imaging element that helps the operator of the catheter determine where the working element is located with respect to tissue.

RELATED APPLICATIONS

This application claims the benefit of priority from U.S. Provisional Patent Application Serial No. 60/241,134, entitled Catheter, filed Oct. 16, 2000. This application is related to U.S. Provisional Patent Application Serial No. 60/245,343, entitled Catheter, filed Nov. 1, 2000; U.S. Provisional Patent Application Serial No. 60/268,264, entitled Catheter, filed Feb. 12, 2001; and U.S. Provisional Patent Application Serial No. 60/268,647, entitled Method and Apparatus for Micro-Dissection of Vascular Occlusions, filed Feb. 13, 2001, all of which are incorporated herein by reference in their entirety.

### FIELD OF THE INVENTION

Embodiments of the invention are in the field of catheters, and more particularly in the field of catheters to be introduced into human vasculature.

### BACKGROUND OF THE INVENTION

In order to treat total or near total occlusions in the vasculature, instruments must be introduced into small body openings and lumens. In many instances, an instrument must navigate a small lumen to a site to be operated on. In such instances, it is necessary for the shaft attached to the working element to be steerable and to have the appropriate flexibility and strength. In some procedures, a working element on the distal end of a catheter is placed in contact with an occlusion in order to make a passage through the occlusion using blunt dissection. The catheter must thus have an appropriate working element and actuation mechanism.

### SUMMARY OF THE DISCLOSURE

Embodiments of a catheter for intravascular procedures are described. Some simplifications and omissions may be made in the following brief summary of some embodiments and aspects of the invention. The summary is intended to highlight and introduce some aspects of the disclosed embodiments, but not to limit the scope of the invention. Thereafter, a detailed description of illustrated embodiments is presented, which will permit one skilled in the relevant art to make and use aspects of the invention. One skilled in the relevant art can obtain a full appreciation of aspects of the invention from the subsequent detailed description, read together with the figures, and from the claims (which follow the detailed description). The embodiments described include a catheter with various elements arranged about a central axis. The elements include an inner shaft, an outer shaft, an actuation mechanism, and a distal working element. In one embodiment, the inner shaft forms the actuation mechanism, and includes a flexible hypotube. The flexible hypotube further forms a lumen that can accommodate, for example, a guidewire. The actuation mechanism actuates, or deploys, a working element at the distal end of the catheter. In one embodiment, the working element includes a tissue spreading member for disrupting an occlusion. In one embodiment, the catheter includes an imaging element that helps the operator of the catheter determine where the working element is located with respect to tissue.

### BRIEF DESCRIPTION OF THE DRAWINGS

FIG. 1 is a perspective view of a distal section of one embodiment of a catheter.

FIG. 2 is a cross-sectional view of an embodiment of a catheter.

FIG. 3 is a side elevation view of an embodiment of a right side of a distal catheter section.

FIG. 4 is a side elevation view of the left side of the distal catheter section shown in FIG. 3.

FIG. 5 is an exploded perspective view of the embodiment of FIG. 3.

FIG. 6 is a perspective view of the link shown removed from the distal catheter section of FIG. 5.

FIG. 7 is an enlarged perspective view of the catheter base sections shown removed from the distal catheter section of FIG. 5.

FIG. 8 is an exploded perspective view of an alternate embodiment of a catheter section.

FIG. 9 is a perspective view of an embodiment of a flexible instrument shaft.

FIG. 10 is a side elevation view of an embodiment of jaws, and a distal base section of a catheter.

FIG. 11 is a perspective view of a distal section of one embodiment of a catheter.

FIG. 12 is a cross-sectional view of an embodiment of a catheter handle assembly with a pivotal control handle portion.

FIG. 13 is a cross-sectional view of an embodiment of a catheter handle assembly with a pivotal control handle portion.

FIG. 14 is a cross-sectional view of an embodiment of a catheter handle assembly with a pivotal control handle portion.

FIG. 15 is a cross-sectional view of an embodiment of a catheter including an imaging device.

### DETAILED DESCRIPTION

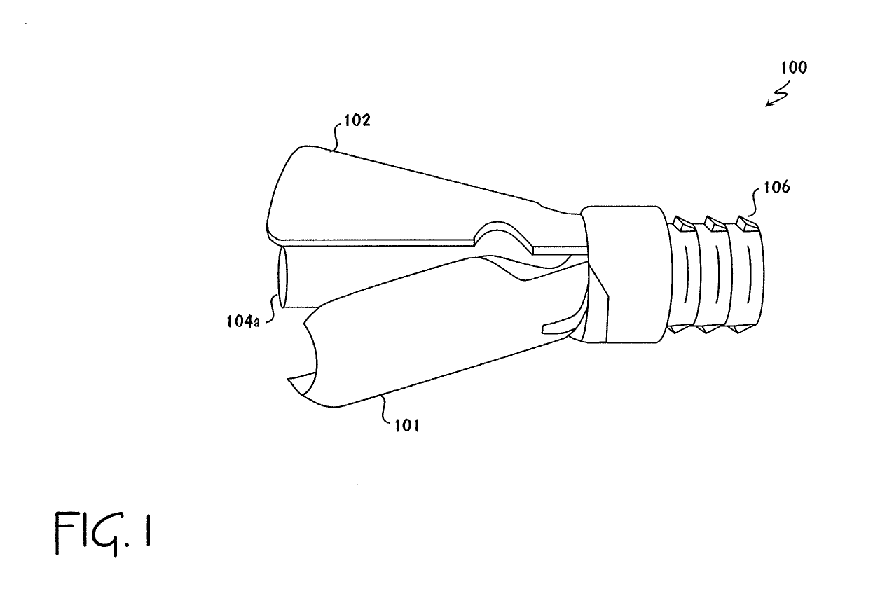

The following description provides specific details for a thorough understanding of, and enabling description for, embodiments of the invention. However, one skilled in the art will understand that the invention may be practiced without these details. In other instances, well known structures and functions have not been shown or described in detail to facilitate the description of the embodiments of the invention. FIG. 1 is a perspective view of a distal section 100 of one embodiment of a catheter. The distal section 100 includes a distal base section 104a, which serves as an actuation member to be more fully described. The distal base section 104a actuates, or deploys, one or more working elements that includes a tissue displacing member, or jaw 101, and a tissue displacing member, or jaw 102. The jaws 101 and 102 are shown in an open position, indicating that the jaws 101 and 102 are at least partially deployed. In the deployed position, the jaws 101 and 102 disrupt tissue with which they come in contact. The jaws 101 and 102 are pivotally attached to a link 106.

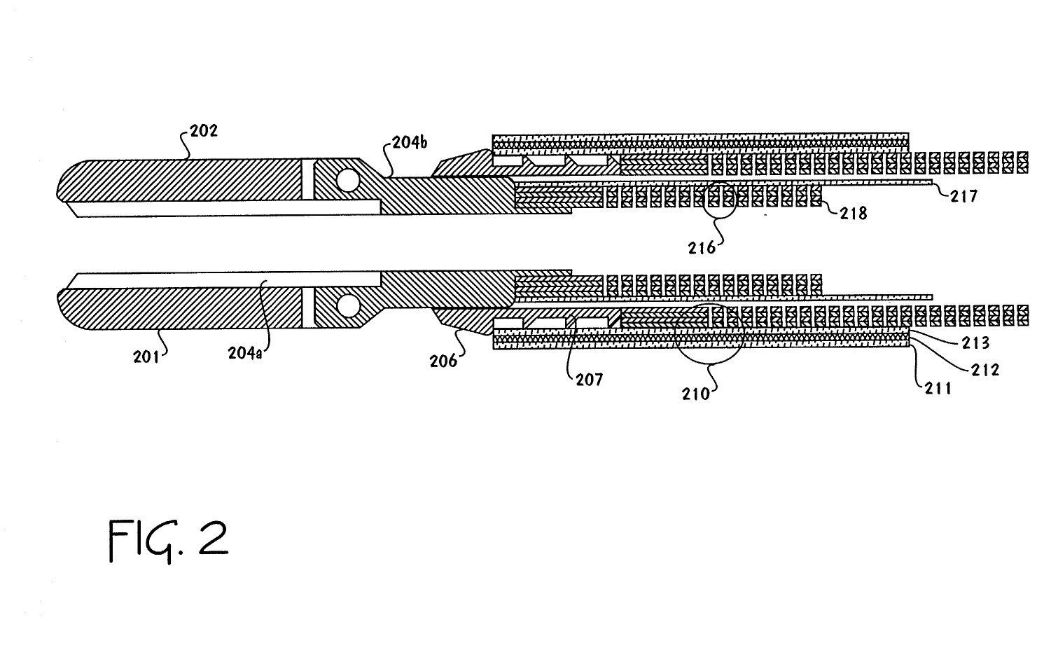

FIG. 2 is a cross-sectional view of an embodiment of a catheter 200 including a distal section similar to the distal section shown in FIG. 1. The catheter 200 includes a working element made up of a pair of jaws 201 and 202. The catheter 200 further includes a distal base section 204a. The jaws 201 and 202 are shown in a closed, or undeployed, position. The jaws 201 and 202 are typically in the undeployed position when the working element is being moved to a site in a patient's body where a procedure is to be performed. For example, the catheter 200 with the jaws 201 and 202 in the undeployed position can be moved through a vasculature occlusion by actuating the jaws 210 and 202 to establish a pathway through the occlusion by dissecting the tissue encountered. The catheter 200 with the jaws 201 and 202 in the undeployed position can also be moved through a subintimal space between tissue layers, or a “false lumen”, in order to go around a total or near total occlusion in a vasculature. The distal base section 204a forms a lumen coaxially aligned with a central axis of the catheter 200. The lumen can accommodate various devices, including a guidewire.

The catheter 200 further includes a proximal base section 204b connected to the distal base section 204a, for example by welding. A link 206 is connected to the proximal base section 204b. In one embodiment, the proximal base section 204b is pressed into the link 206. In another embodiment, the proximal base section 204b is pressed into the link 206 and welded. The link 206 includes ribs 207 for securing a flexible outer shaft 210 to the link 206. In one embodiment, the outer shaft 210 includes an outer polymer layer 211 that forms an outer circumference of the outer shaft 210. An annular braided element 212 is made of a braided material, such as braided steel, and is in contact with the inner diameter of the outer polymer layer 211. An inner polymer layer 213 is in contact with the inner diameter of the braided element 212. Against the inner diameter of the inner polymer layer is a coil 214. In one embodiment, the coil 214 is a high aspect ratio coil of a selected metal alloy that has good flexibility and strength characteristics. A high aspect ratio coil, and other elements of an instrument shaft that is applicable to the catheter described herein, is further described in U. S. patent application Ser. No. 09/812,355, entitled Instrument Shaft, filed Mar. 19, 2001, which is incorporated herein by reference.

The catheter 200 further includes a flexible inner shaft 216. The inner shaft 216, in one embodiment, is pressed and/or welded onto the proximal base section 204b. The inner shaft 216 includes a shaft liner 217 that, in one embodiment, is fabricated of a polymer. The inner shaft 216 further includes a flexible hypotube 218 that is in contact with the inner diameter of the shaft liner 217. Embodiments of a flexible hypotube applicable to the catheter described herein is further described in the U. S. Patent Application entitled Flexible Instrument Shaft, Attorney Docket No. 37217.8065, serial number not yet assigned (filed conccurrently with this application), which is incorporated herein by reference. The inner shaft 216 serves as an actuator for the working element that includes the jaws 201 and 202, as illustrated more fully in further figures. The inner shaft 216, the distal base section 204a, and the proximal base section 204b form a lumen that can accommodate various elements therethrough, such as a guidewire. In various embodiments, the inner shaft 216 and the outer shaft 210 may have different components to provide different physical characteristics. For example, a flexible hypotube such as flexible hypotube 218 with a laminated covering can be used for the outer shaft 210 instead of the coil 214. Any combination of materials and elements is possible to provide characteristics such as stiffness, flexibility, and compressibility as required. The inner and outer shafts 216 and 210 may further be fabricated of a single material, such as Nitinol.

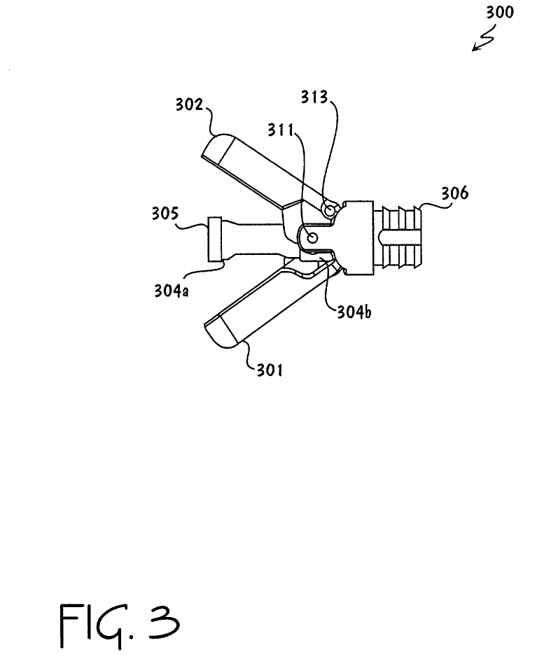

FIG. 3 is a side elevation view of a right side of an embodiment of a distal catheter section 300. FIG. 3 shows a pair of jaws 301 and 302, a distal base section 304a intermediate the jaws 301 and 302, and a link 306. The jaw 302 is pivotally connected to a proximal base section (not completely visible) by a pin 311. The jaw 302 is also pivotally connected to the proximal base section (not completely visible) by a pin 313. The distal base section 304a includes a lip 305 that, in one embodiment, is flush with a liner material (not shown) after the liner material is applied.

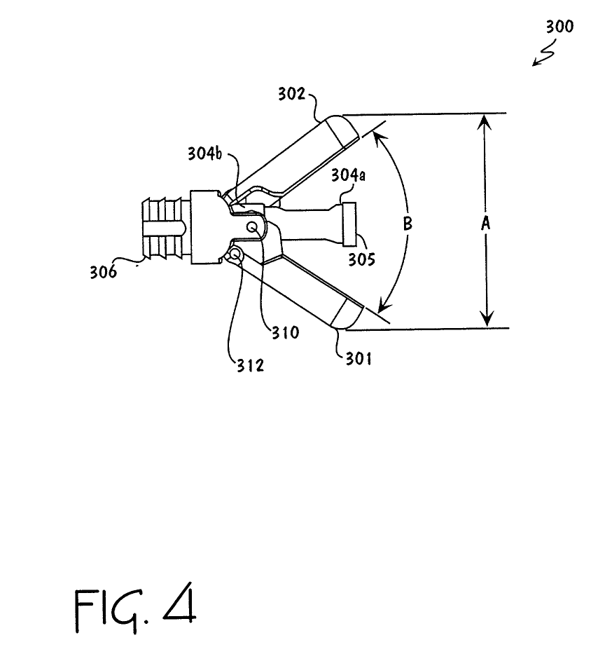

FIG. 4 is a side elevation view of a left side of the catheter section 300. FIG. 4 shows the jaw 301, the jaw 302, the distal base section 304a, and the link 306. The jaw 301 is pivotally connected to the proximal base section 304b (not completely visible) by a pin 310. The jaw 302 is also pivotally connected to the proximal base section 304b (not completely visible) by a pin 312. A dimension A, in one embodiment, represents how far jaws 301 and 302 open in a deployed position, and is in a range of approximately 0.120 inch to 0.200 inch inclusive.

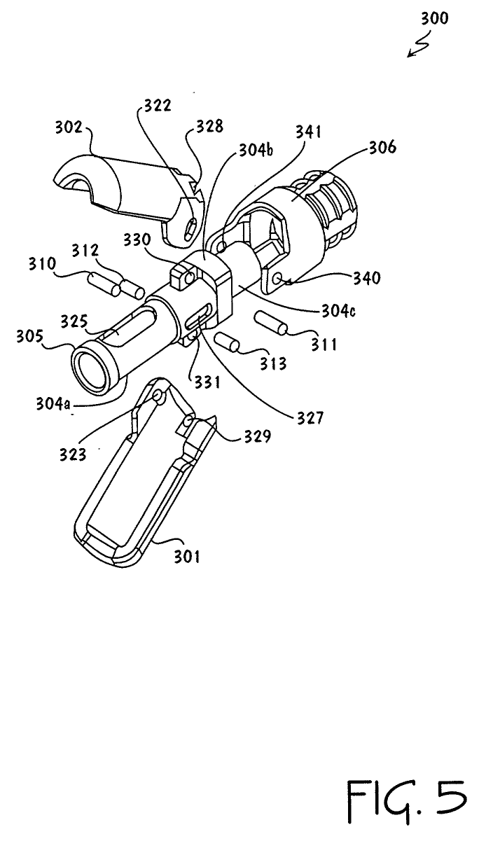

FIG. 5 is an exploded view of the catheter section 300. The jaws 301 and 302, the distal base section 304a, and the link 306 are shown. The proximal base section 304b is visible in the figure. In one embodiment, the proximal base section 304b and the distal base section 304a are separately formed components that are bonded together using a technique appropriate to the material. The distal base section 304a, in one embodiment, includes an opening 325. An opening corresponding to the opening 325 (not shown) is on the opposite side of the distal base section 304a. The opening 325 provides an area for bonding a liner (not shown), such as a polymer liner, from the inner diameter to the outer diameter of the distal base section 304a. This provides a mechanical lock to maintain the inner liner in position within the distal base section 304a and the proximal base section 304b. The lip 305 is substantially flush with an outer surface of the liner after the liner is applied. An ear of the jaw 302 includes a hole 322 for receiving the pin 313, which also goes through a hole in the side of the proximal base 304b. The pin 313 is also slidably disposed in the slot 327. A slot corresponding to the slot 327 (not shown) is located 180 degrees away from the slot 327, and receives the pin 312. The pin 311 goes through a hole 328 in top of the jaw 302, and also through hole 330 in the proximal base section 304b. The jaw 301 is correspondingly connected to the proximal base section 304b by the pins 312 and 310. For example, the pin 312 goes through the holes 341, 323, and 322, and the pin 310 goes through a hole 329 in the top of the jaw 301 and hole 331 in the proximal base section 304b.

An inner shaft (not shown for clarity), similar to the inner shaft 216 of FIG. 2, is pressed onto and affixed to a proximal-most base section 304c. The inner shaft and the base sections guidewire. The inner shaft and the base sections 304a, 304b, and 304c also form an actuator that deploys the jaws 301 and 302. The inner shaft is connected at a proximal end of a catheter assembly to a manual control, such as a handle. The manual control allows the operator to move the inner shaft and the base section 304 back and forth along a central axis of the catheter. The link 306, which is attached to the catheter shaft, remains stationary while the inner shaft and base sections 304a, 304b, and 304c move. The slots, such as 327 and its corresponding slot (not shown), allow the base to be moved proximally and distally while simultaneously allowing the jaws 301 and 302 to pivot axially about the pins 311 and 312, which translate within the slots. As the base sections 304 move proximally, the jaws 301 and 302 move radially outward from the central axis of the catheter to a deployed position. Conversely, as the base sections 304 move distally, the jaws 301 and 302 move radially inward toward the central axis of the catheter to an undeployed position. This allows the operator to manipulate the jaws 301 and 302 to disrupt tissue and make a passage through an occlusion itself, or between the occlusion and the vessel wall, or within the vessel wall itself, to reach a location distal to the occlusion in the vasculature.

The actuation mechanism, including the inner shaft and the sections 304a, 304b, and 304c, is relatively unexposed to the surface of the catheter. The jaws 301 and 302 present a relatively smooth surface to a body lumen as the catheter negotiates the lumen. The actuation mechanism, including the inner shaft, is pulled or pushed by a manual control at the proximal end of a catheter assembly. Thus, the actuation mechanism is relatively simple, with few moving parts and few mechanical fasteners as compared, for example, to a catheter that has both a guidewire lumen and a separate actuation wire in the guidewire lumen or in another lumen.

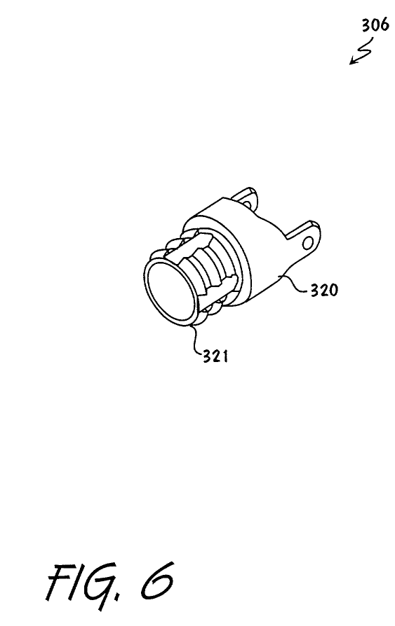

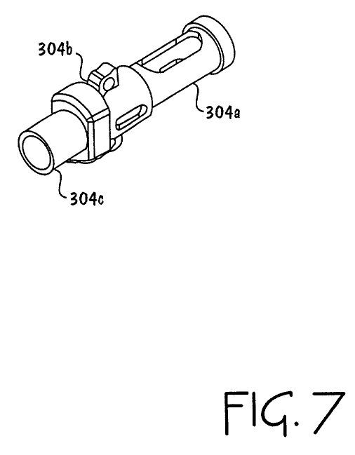

FIG. 6 is a perspective view of a link 306, including a proximal ribbed section 321 and a distal section 320 with ears. The ears of the distal section 320 accept pins as described previously. FIG. 7 is a perspective view of the base sections 304a, 304b and 304c, as previously described.

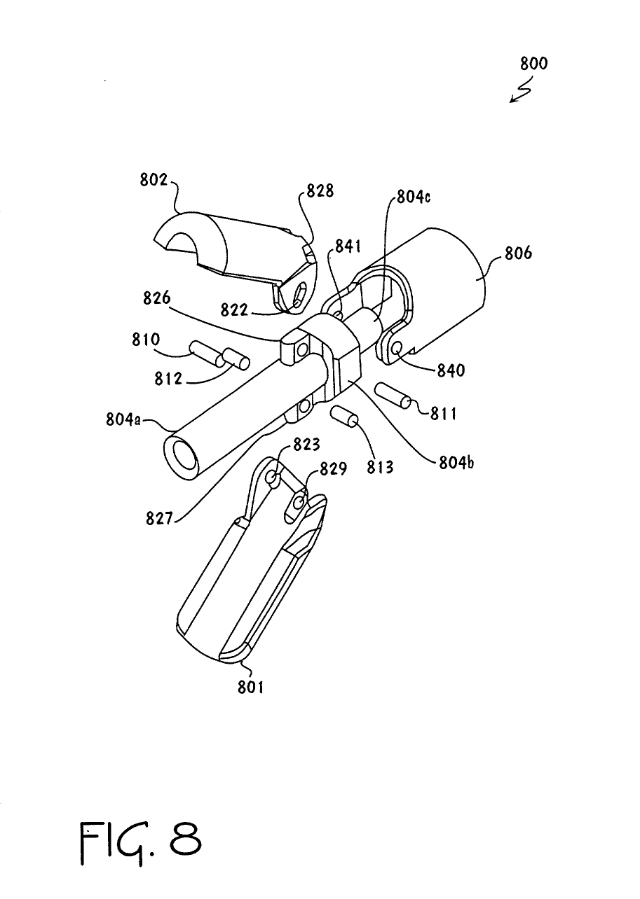

FIG. 8 is an exploded view of an embodiment of a distal catheter section 800. Jaws 801 and 802, the distal base section 804a, and a link 806 are shown. A proximal base section 804b and a proximal-most base section 804c are also shown. In one embodiment, the proximal base section 804b and the distal base section 804a are separately formed components that are bonded together, for example metal components welded together. An ear 826 includes a hole for receiving the pin 811, which also goes through a hole 828 in the top of the jaw 802. The pin 813 goes through a hole 840 in the link 806, through a slot 822 in the jaw 802, and internally rests against the distal base section 804b. The jaw 801 is correspondingly connected to the proximal base section 804b by the pins 810 and 812. For example, the pin 810 goes through the hole 829, and the hole shown in the ear 827. The pin 812 goes through a hole 841 in the link 806, and through the slot 823.

An inner shaft (not shown for clarity), similar to the inner shaft 216 of FIG. 2, is pressed onto and affixed to a proximal-most base section 804c. The inner shaft and the base sections 804a, 804b, and 804c form a lumen through which various components can be passed, such as a guidewire. The inner shaft and the base sections 804 also form an actuator that deploys the jaws 801 and 802. The inner shaft is connected at a proximal end of a catheter assembly to a manual control, such as a handle. The manual control allows the operator to move the inner shaft and the base section 804 back and forth along a central axis of the catheter. The link 806, which is attached to the catheter shaft, remains stationary while the inner shaft and base sections 804 move. The interior ends of the pins 812 and 813 move freely against the distal base section 804a, and simultaneously allow the jaws 801 and 802 to pivot axially about the pins 810 and 811. As the base sections 804 move proximally, the jaws 801 and 802 move radially outward from the central axis of the catheter to a deployed position. Conversely, as the base sections 804 move distally, the jaws 801 and 802 move radially inward toward the central axis of the catheter to an undeployed position. This allows the operator to manipulate the jaws 801 and 802 to disrupt tissue and make a passage through an occlusion itself, or between the occlusion and the vessel wall, or within the vessel wall itself to reach a location distal to the occlusion in the vasculature. The actuation mechanism, including the inner shaft and the base section 804, is relatively unexposed to the surface of the catheter. The jaws 801 and 802 present a relatively smooth surface to a body lumen as the catheter negotiates the lumen. The inner shaft is pulled or pushed by a manual control at the proximal end of a catheter assembly to deploy the jaw 801 and 802. Thus, the actuation mechanism is relatively simple, with few moving parts and few mechanical fasteners as compared, for example, to a catheter that has both a guidewire lumen and a separate actuation wire in the guidewire lumen or in another lumen.

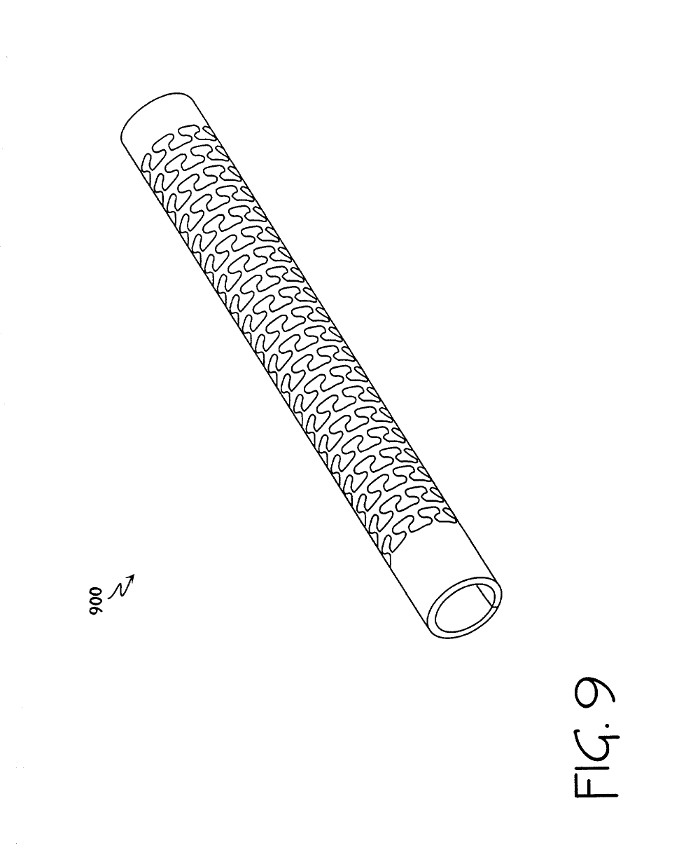

FIG. 9 is a perspective view of a flexible hypotube 900. The flexible hypotube 900 is an embodiment that can be used as the flexible hypotube 218 of FIG. 2. The flexible hypotube 900 can be used as an inner catheter shaft, an outer catheter shaft, or in combination with other materials. The flexible hypotube 900 includes annular sections that each has a uniform pattern on a distal edge and a proximal edge. The annular sections interlock with each other as shown. The annular sections can move with respect to each other as limited by a space between them. The annular sections may optionally be joined at particular places by, for example a spot weld, to limit the amount of flexibility, for example in a particular plane or planes. Embodiments of a flexible hypotube applicable to the catheter described herein is further described in the U.S. Patent Application entitled Flexible Instrument Shaft, Attorney Docket No. 37217.8065, serial number not yet assigned (filed conccurrently with this application), which is incorporated herein by reference.

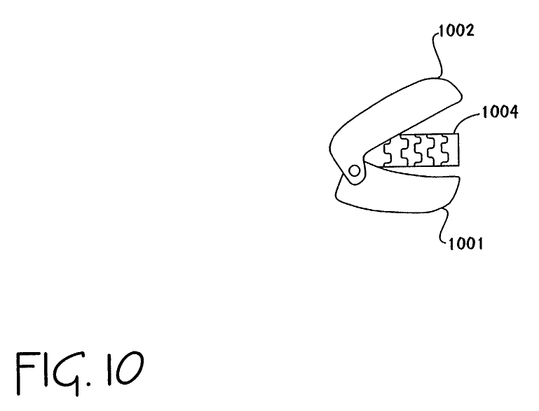

FIG. 10 is a perspective view of a pair of jaws 1001 and 1002, and a distal base section 1004. The distal base section 1004 includes a flexible hypotube similar to the flexible hypotube 900. The distal base section 1004 thus has improved flexibility, which is useful for various functions, for example for providing a lumen for a guidewire.

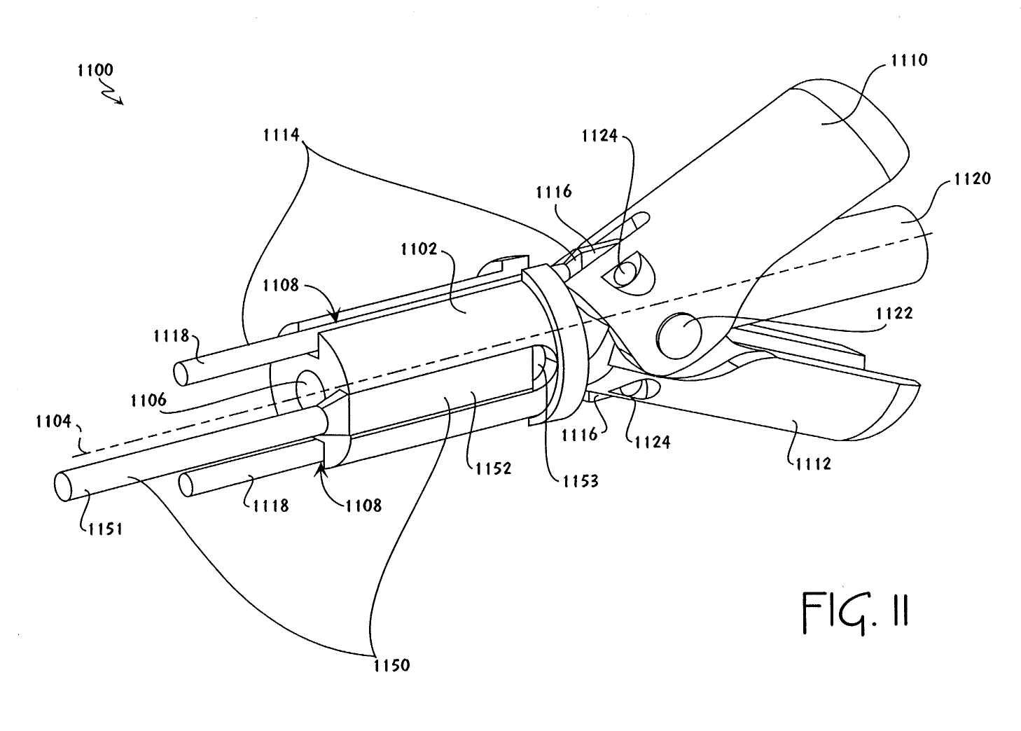

FIG. 11 is a perspective view of an alternative embodiment 1100 of a catheter section. Referring to FIG. 11, the catheter section 1100 includes a base section 1102 having a central axis 1104, a lumen 1106, two actuation channels 1108, and a steering channel 1153. The catheter section 1100 further includes a pair of jaws 1110 and 1112, and two actuation assemblies including an actuation plate 1116 and actuation member 1118. A hinge pin 1122 and a corresponding hinge pin 180 degrees away (not shown) moveably attach the jaws 1110 and 1112 to the base 1102. Coupling pins 1124 attach respective jaws 1110 and 1112 to the actuation plates 1116.

A steering assembly 1150 includes a steering member 1151 and a steering plate 1152. The steering channel 1153 accommodates the steering plate 1152, such that the steering plate 1152 is pressed into and affixed to the base section 1102. The steering plate 1152 is bonded to the base section 1102 in one embodiment, for example by welding or adhesive bonding. The steering member 1151 is bonded to the steering plate 1152. Alternatively, the steering member 1151 and the steering plate 1152 are produced as one piece.

Pulling or pushing the steering member imparts a moment about the assembly immediately proximal to the base 1102, and bends the assembly about the axis 1104. The central axis 1104 extends through the base section 1102 and through the lumen 1106. The lumen 1106 begins at the proximal end of the base 1102 and continues through to the distal end of the catheter section 1100. The lumen can accommodate a guidewire, catheter, or other intervention device.

In operation, the catheter section 1100 is placed into approximate contact with a vascular occlusion and/or a blood vessel wall to facilitate the disruption of the vascular occlusion. This placement can be controlled by the steering assembly 1150, or the assembly can be tracked to the site over a guidewire placed in the lumen 1106. The application of a force in the proximal or distal direction of the steering member while not advancing the catheter displaces the apparatus laterally to facilitate the proper positioning relative to the occlusion. In other embodiments the catheter section may comprise more than one steering assembly.

An actuation force is applied independently to either actuation assembly 1114 through actuation member 1118, which has the effect of opening a corresponding jaw 1110 or 1112. The jaws 1110 and 1112 can be operated independently. The jaws 1110 and 1112 are open when they are displaced from the central axis of the catheter section. When the jaws 1110 and 1112 are opened in contact with tissue or an occlusion, the jaws tear, fracture or otherwise disrupt the tissue or occlusion.

The embodiment of FIG. 11 is further described in U.S. provisional patent application serial number 60/268,647, entitled Method and Apparatus for Micro-Dissection of Vascular Occlusions, filed Feb. 13, 2001, and incorporated herein by reference.

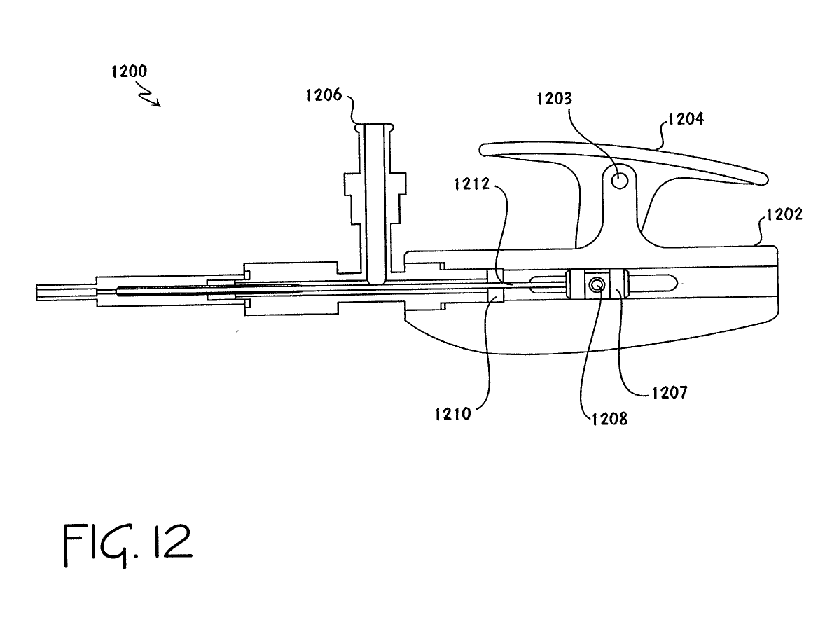

FIG. 12 is a cross-sectional view of an embodiment of a handle assembly 1200 usable on the proximal end of a catheter assembly. For example, the handle assembly 1200 is usable with the catheter sections shown in FIG. 2 and in FIGS. 3-7. The handle assembly 1200 is a manual control that allows an operator to deploy the jaws of the catheter section 300 during a procedure. The handle assembly includes a control handle 1204 pivotally attached to a handle body 1202 through a pin 1203. A base of the handle 1204 is pivotally attached to a sliding member 1207 slidably disposed in an axial bore extending through the handle body 1202. The handle 1204 is pivotally connected to a pin 1208 on each side of the sliding member 1207. The pin 1208 does not penetrate and inner diameter of the sliding member 1207, thus leaving the interior of the sliding member open to accommodate other elements. A corresponding pin (not shown) attaches the handle 1204 to the sliding member 1207 on the side of the catheter assembly that is not shown. An actuation member 1212 is shown attached to the sliding member 1207. In one embodiment, the actuation member is similar to item 216 of FIG. 2.

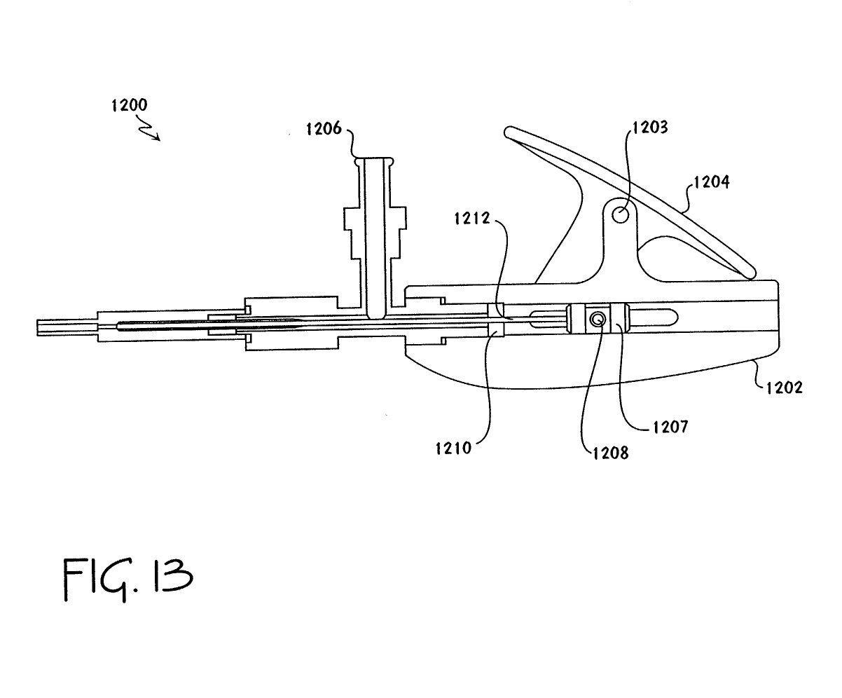

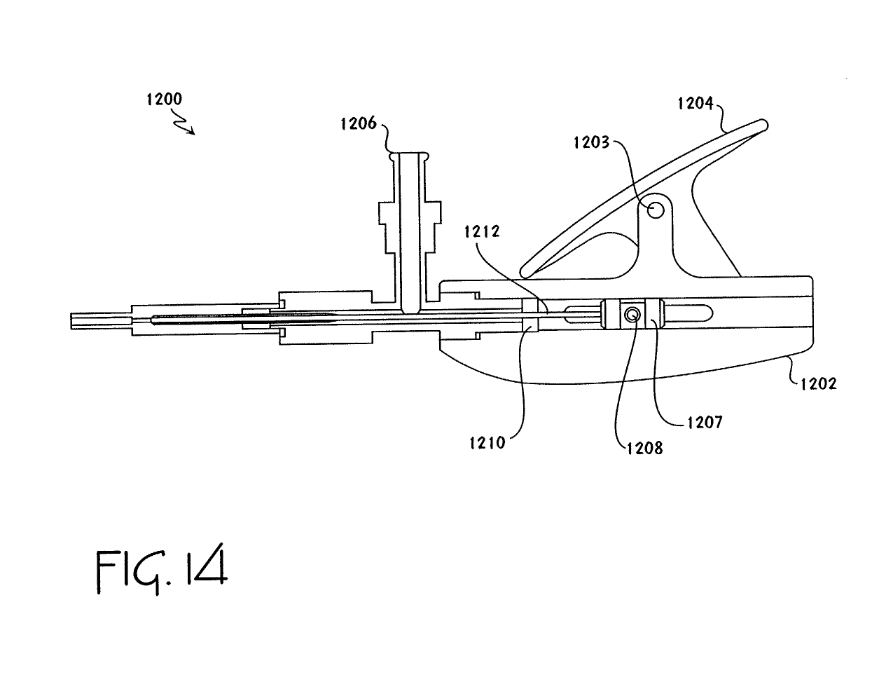

The handle assembly 1200 further includes a flush port 1206. Referring to the embodiment of FIG. 2, the flush port 1206 would communicate with the annular space between assembly 216 and outer shaft 210. The handle assembly further includes a stop 1210 that assists in limiting the distal movement of the actuation mechanism, such as the actuation mechanism including a flexible inner shaft 216 shown in FIG. 2. Different positions of the handle 1204 and the resulting movement of catheter components is illustrated in FIG. 13 and 14. FIG. 13 is a cross-sectional view of the handle assembly 1200 in an undeployed condition in which the jaws (for example jaws 401 and 402) are fully closed. The stop 1210 limits the distal movement of the sliding member 1207. In addition, the handle 1204 contacts the handle body 1202 in the fully closed position, limiting the movement of the sliding member 1207. FIG. 14 is a cross-sectional view of the handle assembly 1200 in a deployed condition in which the jaws (for example jaws 401 and 402) are fully open. The handle 1204 contacts the handle body 1202 in the fully open position, limiting the proximal movement of the sliding member 1207.

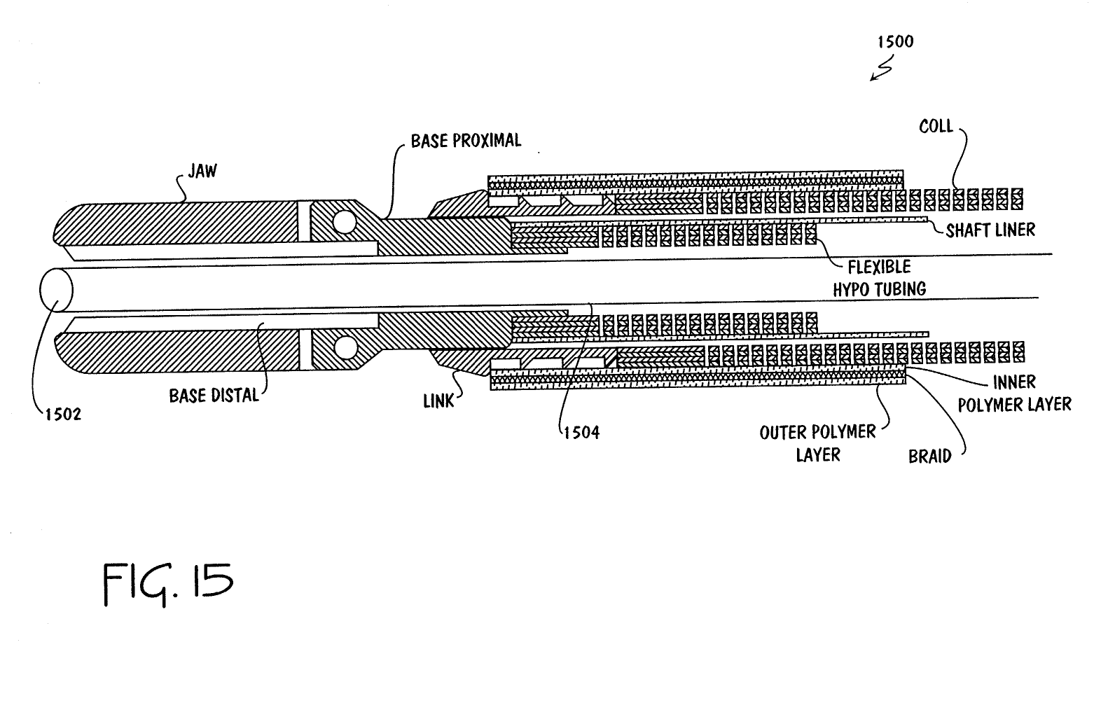

FIG. 15 is a cross-sectional view of an embodiment including an imaging device 1502 included in a catheter 1500 which is similar to the embodiment of FIG. 2. The imaging device, in one embodiment, uses optical coherence tomography (OCT). An OCT system delivers infrared (IR) light into tissue at the distal end of a rotating optical fiber 1502. In one embodiment, the optical fiber 1502 is covered by a polyamide sleeve 1504. Delivery of the light into the tissue is accomplished by terminating the optical fiber at an angle, for example 45 degrees, to achieve internal reflection of the light at an approximate right angle to the central axis of the optical fiber. The optical fiber also receives reflection of the light from various tissue types. The reflected light signals are deciphered and a cross-sectional image of the tissue surrounding the tip of the optical fiber is produced. In general, OCT measures the intensity of back-reflected IR light and allows resolution 5 to 25 times greater than current ultrasound techniques. Currently, OCT cannot be used to reliably generate an image through blood. However, the wavelength of the IR light may be varied to pass through blood and produce an image. Blood and other fluids can also be evacuated from the area of interest.

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” and words of similar import, when used in this application, shall refer to this application as a whole and not to any particular portions of this application.

The above description of illustrated embodiments of the invention is not intended to be exhaustive or to limit the invention to the precise form disclosed. While specific embodiments of, and examples for, the invention are described herein for illustrative purposes, various modifications are possible within the scope of the invention, as those skilled in the relevant art will recognize. For example, a working element that includes a different working element than those shown, such as a single jaw or an imaging device without jaws, is within the scope of the invention. The elements and acts of the various embodiments described above can be combined to provide further embodiments beyond those described herein.

These and other changes can be made to the invention in light of the above detailed description. In general, in the following claims, the terms used should not be construed to limit the invention to the specific embodiments disclosed in the specification and the claims. Accordingly, the invention is not limited by the disclosure, but instead the scope of the invention is to be determined entirely by the claims.

While certain aspects of the invention are presented below in certain claim forms, the inventors contemplate the various aspects of the invention in any number of claim forms. Accordingly, the inventors reserve the right to add additional claims after filing the application to pursue such additional claim forms for other aspects of the invention.

What is claimed is:

1. An apparatus for treating a vascular occlusion, comprising:
a flexible outer shaft about a central lumen of the apparatus;
a flexible inner shaft about the central lumen;
a distal working element about the central lumen for treating the vascular occlusion, wherein the distal working element is coupled to the flexible inner shaft through at least one coupling member; and
a manual control coupled to a proximal end of the apparatus and to the flexible inner shaft such that the manual control is manipulable to deploy the distal working element via the flexible inner shaft.

2. The apparatus of claim 1, wherein the at least one coupling member includes a link coupled to the flexible outer shaft and a base coupled to the link and to the inner flexible shaft, wherein the manual control is manipulable to move the inner flexible shaft distally and proximally along a central axis of the apparatus.

3. The apparatus of claim 2, wherein the base includes:
a proximal-most base section, wherein the inner flexible shaft is pressed onto the proximal-most base section; and
a proximal base section, wherein the working element is pivotally coupled to the proximal base section.

4. The apparatus of claim 3, wherein the at least one coupling member further includes a link, wherein the link is slidably coupled to the base and to the working element such that the working element is deployed when the inner flexible shaft and the base are translated proximally with respect to the link.

5. The apparatus of claim 4, wherein the working element includes at least one jaw that is pivotally coupled to the base at one location and slidably coupled to the base at another location such that a distal end of the at least one jaw moves radially away from the central axis when the working element is deployed.
